(19) United States
(12) Patent Application Publication
(10) Pub. No.: US 2010/0063074 A1
(43) Pub. Date: Mar. 11, 2010

(54) Cancer Treatment Method

(76) Inventors: Mark S. Berger, Merion Station, PA (US); Tona Morgan Gilmer, Durham, NC (US); Arundathy Nirmalini Pandite, Durham, NC (US); David Rusnak, Durham, NC (US); Neil L. Spector, Durham, NC (US); Iman El-Hariry, Greenford (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/569,877

(22) PCT Filed: Jun. 1, 2005
(86) PCT No.: PCT/US05/19053

§ 371 Date: Sep. 21, 2009

Related U.S. Application Data
(60) Provisional application No. 60/576,825, filed on Jun. 3, 2004.
(60) Provisional application No. 60/606,790, filed on Sep. 2, 2004.

(51) Int. Cl. A61K 31/517; A61P 35/00

(52) U.S. Cl. 514/266.24

(57) ABSTRACT

The present invention relates to a method of treating cancer in a mammal by administration of 4-quinazolinamines and pharmaceutical compositions containing the same. In particular, the method relates to a methods of treating cancers which are mediated by the tyrosine kinases EGFR and/or erbB2 by administration of N-{3-chloro-4-[(3-fluorobenzyl)oxy]phenyl}-6-[5-({[2-(methanesulphonyl)ethyl]amino}methyl)-2-furyl]-4-quinazolinamine and salts and solvates thereof.

### BACKGROUND OF THE INVENTION

The present invention relates to a method of treating cancer in a mammal by administration of 4-quinazolinamines and pharmaceutical compositions containing the same. In particular, the method relates to methods of treating cancers which are mediated by the tyrosine kinases EGFR and/or erbB2 by administration of N-{3-chloro-4-[(3-fluorobenzyl)oxy]phenyl}-6-[5-({[2-(methanesulphonyl)ethyl]amino}methyl)-2-fury/]-4-quinazolinamine and salts and solvates thereof.

Effective chemotherapy for cancer treatment is a continuing goal in the oncology field. Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993). One particular pathway of note is cellular signalling from the growth factor receptors of the erbB family.

There is significant interaction among the erbB family that regulates the cellular effects mediated by these receptors. Six different ligands that bind to EGFR include EGF, transforming growth factor, amphiregulin, heparin binding EGF, betacellulin and epiregulin (Alroy & Yarden, FEBS Letters, 410:83-86, 1997; Burden & Yarden, Neuron, 18: 847-855, 1997; Klapper et al., ProcNatlAcadSci, 4994-5000, 1999). Heregulins, another class of ligands, bind directly to HER3 and/or HER4 (Holmes et al., Science, 256:1205, 1992; Klapper et al., 1997, Oncogene, 14:2099-2109; Peles et al., Cell, 69:205, 1992). Binding of specific ligands induces homo- or heterodimerization of the receptors within members of the erbB family (Carraway & Cantley, Cell, 78:5-8, 1994; Lemmon & Schlessinger, TrendsBiochemSci, 19:459-463, 1994). In contrast with the other ErbB receptor members, a soluble ligand has not yet been identified for HER2, which seems to be transactivated following heterodimerization. The heterodimerization of the erbB-2 receptor with the EGFR, HER3, or HER4 receptors is preferred to homodimerization (Klapper et al., 1999; Klapper et al., 1997). Receptor dimerization results in binding of ATP to the receptor's catalytic site, activation of the receptor's tyrosine kinase, and autophosphorylation on C-terminal tyrosine residues. The phosphorylated tyrosine residues then serve as docking sites for proteins such as Grb2, Shc, and phospholipase C, that, in turn, activate downstream signaling pathways, including the Ras/MEK/Erk and the PI3K/Akt pathways, which regulate transcription factors and other proteins involved in biological responses such as proliferation, cell motility, angiogenesis, cell survival, and differentiation (Alroy & Yarden, 1997; Burgering & Coffer, Nature, 376:599-602, 1995; Chan et al., AnnRevBiochem, 68:965-1014, 1999; Lewis et al., AdvCanRes, 74:49-139, 1998; Liu et al., Genes and Dev, 13:786-791, 1999; Muthuswamy et al., Mol&CellBio, 19, 10:6845-6857, 1999; Riese & Stern, Bioessays, 20:41-48, 1998).

Several strategies including monoclonal antibodies (Mab), immunoconjugates, anti-EGF vaccine, and tyrosine kinase inhibitors have been developed to target the erbB family receptors and block their activation in cancer cells (reviewed in (Sridhar et al., Lancet, 4, 7:397-406, 2003)). Because erbB2-containing heterodimers are the most stable and preferred initiating event for signaling, interrupting both erbB2 and EGFR simultaneously is an appealing therapeutic strategy. A series of 6-furanyiquinazoline dual erbB-2/EGFR TK inhibitors that possess efficacy in pre-clinical models for cancer have been synthesized (Cockerill et al., BiorgMedChemLett, 11:1401-1405, 2001; Rusnak et al., CanRes, 61:7196-7203, 2001a; Rusnak et al., MolCanTher, 1:85-94, 2001b). GW572016 is a 6-furanyiquinazoline, orally active, reversible dual kinase inhibitor of both EGFR and erbB2 kinases (Rusnak et al., 2001b). In human xenograft studies, GW572016 has shown dose-dependent kinase inhibition, and selectively inhibits tumor cells overexpressing EGFR or erbB2 (Rusnak et al., 2001b; Xia et al., Oncogene, 21:6255-6263, 2002).

The present inventors have now identified novel cancer treatment methods which includes administration of N-{3-chloro-4-[(3-fluorobenzyl) oxy]phenyl}-6-[5-({[2-(methanesulphonyl)ethyl]amino}methyl)-2-furyl]-4-quinazolinamine (GW572016) as well as salts and/or solvates thereof.

### SUMMARY OF THE INVENTION

In a first aspect of the present invention, there is provided a method of treating an EGFR and/or erbB2 overexpressing cancer in a mammal, comprising: administering to said mammal therapeutically effective amounts of a compound of formula (I″)

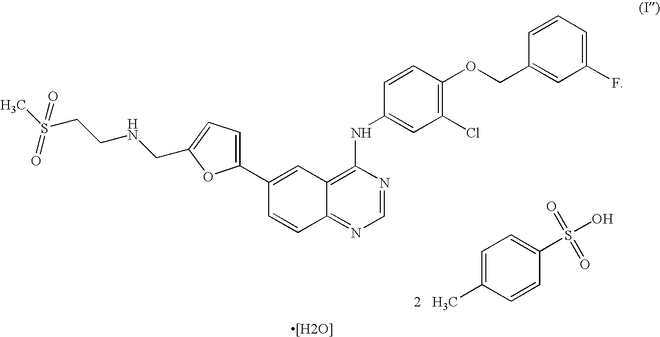

In a second aspect of the present invention, there is provided a method of treating an EGFR overexpressing cancer in a mammal, comprising: administering to said mammal therapeutically effective amounts of a compound of formula (I″)

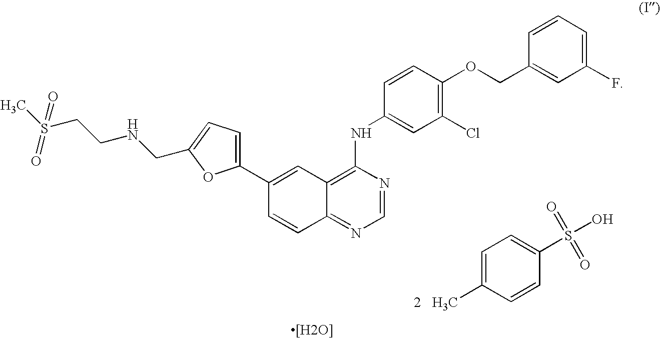

In a third aspect of the present invention, there is provided a method of treating an erbB2 overexpressing cancer in a mammal, comprising: administering to said mammal therapeutically effective amounts of a compound of formula (I″)

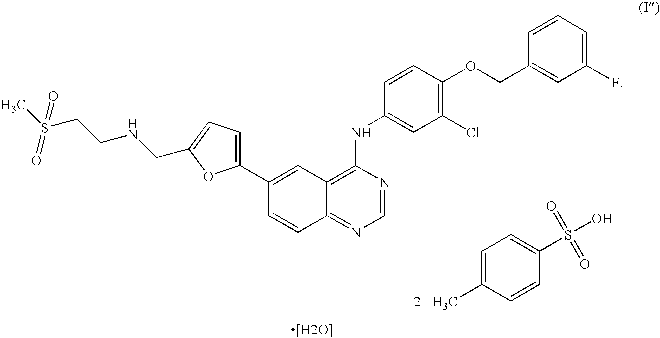

In a fourth aspect of the present invention, there is provided a method of treating an EGFR and erbB2 overexpressing cancer in a mammal, comprising: administering to said mammal therapeutically effective amounts of a compound of formula (I″)

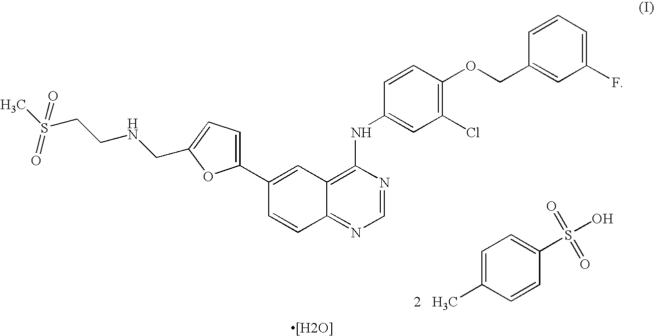

In a fifth aspect of the present invention, there is provided a method of treating renal cancer in a mammal, comprising: administering to said mammal therapeutically effective amounts of a compound of formula (I)

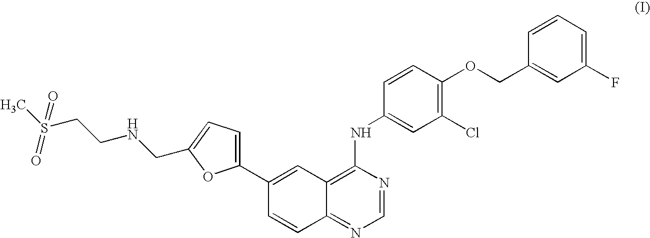

or salts or solvates thereof.

In a sixth aspect of the present invention, there is provided a method of treating bladder cancer in a mammal, comprising: administering to said mammal therapeutically effective amounts of a compound of formula (I)

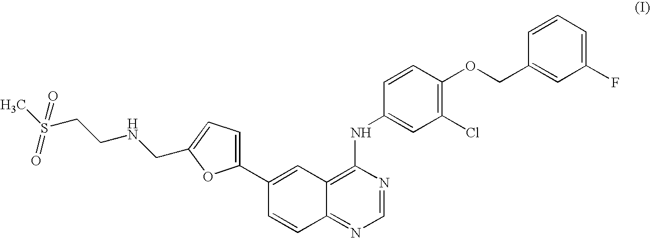

or salts or solvates thereof.

### DETAILED DESCRIPTION OF THE INVENTION

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As is well known in the art, tumors are frequently metastatic, in that a first (primary) locus of tumor growth spreads to one or more anatomically separate sites. As used herein, reference to “a tumor” in a subject includes not only the primary tumor, but metastatic tumor growth as well.

“EGFR”, also known as “erbB-1”, and “erbB-2” are protein tyrosine kinase transmembrane growth factor receptors of the erbB family. Protein tyrosine kinases catalyse the phosphorylation of specific tyrosyl residues in various proteins involved in the regulation of cell growth and differentiation (A. F. Wilks, Progress in Growth Factor Research, 1990, 2, 97-111; S. A. Courtneidge, Dev. Supp.l, 1993, 57-64; J. A. Cooper, Semin. Cell Biol., 1994, 5(6), 377-387; R. F. Paulson, Semin. Immunol., 1995, 7(4), 267-277; A. C. Chan, Curr. Opin. Immunol., 1996, 8(3), 394-401). The ErbB family of type I receptor tyrosine kinases includes ErbB1 (also known as the epidermal growth factor receptor (EGFR or HER1)), erbB2 (also known as Her2), erbB3, and erbB4. These receptor tyrosine kinases are widely expressed in epithelial, mesenchymal, and neuronal tissues where they play a role in regulating cell proliferation, survival, and differentiation (Sibilia and Wagner, Science, 269: 234 (1995); Threadgill et al., Science, 269: 230 (1995)). Increased expression of wild-type erbB2 or EGFR, or expression of constitutively activated receptor mutants, transforms cells in vitro (Di Fiore et al., 1987; DiMarco et al, Oncogene, 4: 831 (1989); Hudziak et al., Proc. Natl. Acad. Sci. USA., 84:7159 (1987); Qian et al., Oncogene, 10:211 (1995)). Increased expression of erbB2 or EGFR has been correlated with a poorer clinical outcome in some breast cancers and a variety of other malignancies (Slamon et al., Science, 235: 177 (1987); Slamon et al., Science, 244:707 (1989); Bacus et al, Am. J. Clin. Path, 102:S13 (1994)).

As used herein, a cell “overexpressing” EGFR and/or erbB2 refers to a cell having a significantly increased number of functional EGFR and/or erbB2 receptors, compared to the average number of receptors that would be found on a cell of that same type. For instance, overexpression of EGFR and/or erbB2 has been documented in various cancer types, including breast (Verbeek et al., FEBS Letters 425:145 (1998); colon (Gross et al., Cancer Research 51:1451 (1991)); lung (Damstrup et al., Cancer Research 52:3089 (1992), renal cell (Stumm et al, Int. J. Cancer 69:17 (1996), Sargent et al., J. Urology 142: 1364 (1989)) and bladder (Chow et al., Clin. Cancer Res. 7:1957 (2001); Bue et al., Int. J. Cancer, 76:189 (1998); (Mellon J, Lunec J., J Uro 1996; 155:321-6; Orlando C, Sestini R., J Urol 1996; 156:2089-2093), Turkeri et al., Urology 51: 645 (1998)). Overexpression of ErbB2 may be assessed by any suitable method as is known in the art, including but not limited to imaging, gene amplification, number of cell surface receptors present, protein expression, and mRNA expression. See e.g., Piffanelli et al., Breast Cancer Res. Treatment 37:267 (1996).

As used herein, the term “solvate” refers to a complex of variable stoichiometry formed by a solute (in this invention, compounds of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

The methods of cancer treatment disclosed herein, includes administering a compound of formula (I):

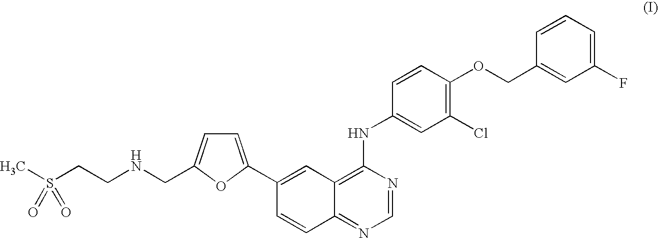

or salts or solvates thereof.

In another embodiment, the compound is a compound of formula (I′) or anhydrate or hydrate forms thereof. The compound of formula (I′) is the ditosylate salt of the compound of formula (I).

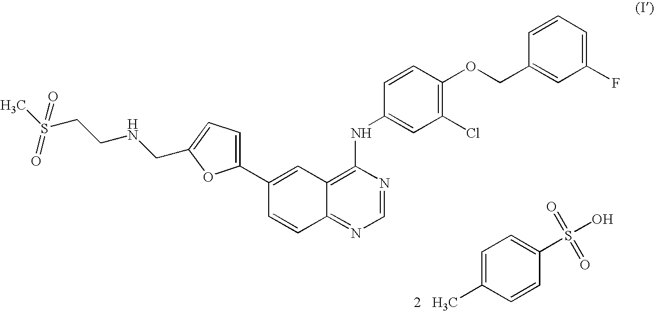

In one embodiment, the compound is the anhydrous ditosylate salt of the compound of formula (I′). In another embodiment, the compound is a compound of formula (I″) which is the monohydrate ditosylate salt of the compound of formula (I′).

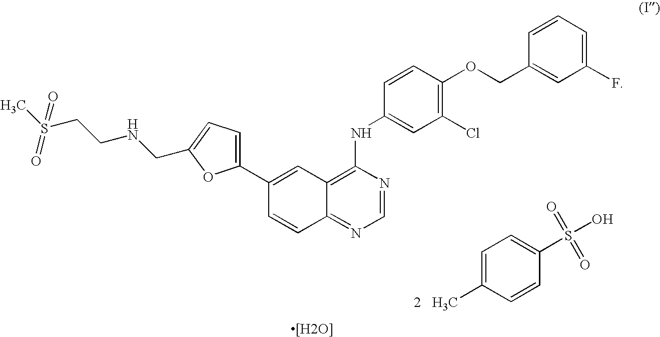

The monohydrate ditosylate salt of the compound of formula (I) has the chemical name N-{3-chloro-4-[(3-fluorobenzyl)oxy]phenyl}-6-[5-({[2-(methanesulphonyl)ethyl]amino}methyl)-2-furyl]-4-quinazolinamine (GW572016) ditosylate monohydrate and is also known as lapatinib.

The free base, HCl salts, and ditosylate salts of the compound of Formula (I) may be prepared according to the procedures of International Patent Application No. PCT/EP99/00048, filed Jan. 8, 1999, and published as WO 99/35146 on Jul. 15, 1999, referred to above and International Patent Application No. PCT/US01/20706, filed Jun. 28, 2001 and published as WO 02/02552 on Jan. 10, 2002 and according to the appropriate Examples recited below. One such procedure for preparing the ditosylate salt of the compound of formula (I) is presented following in Scheme 1.

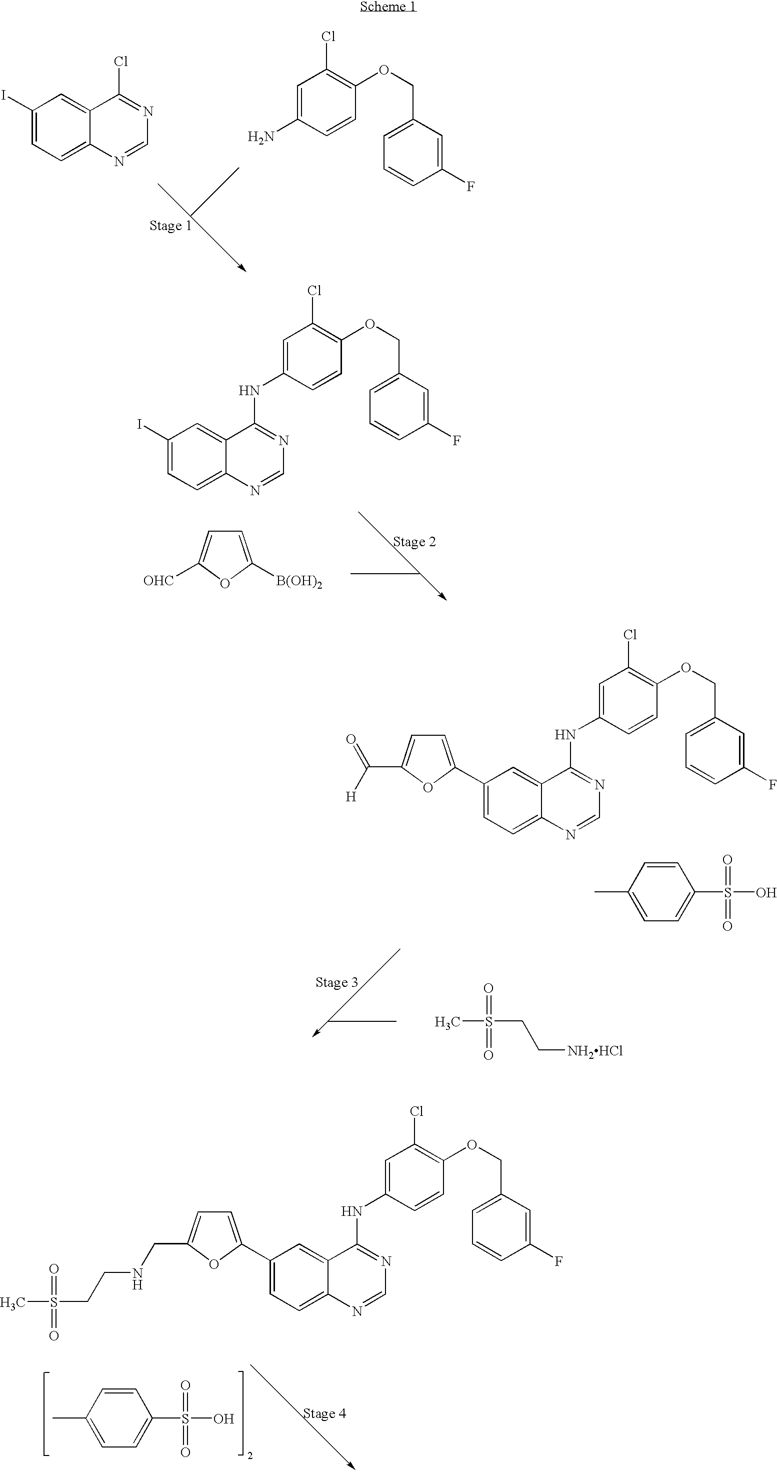

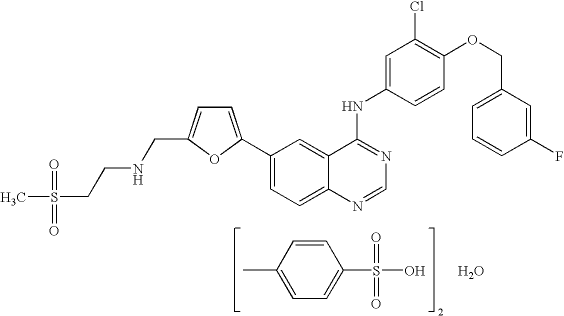

In scheme 1, the preparation of the ditosylate salt of the compound of formula (III) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonase, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in the cancer treatment methods of the present invention therapeutically effective amounts of a compound of formula (I) as well as salts or solvates thereof, may be administered as the raw chemical, it is possible to present the active ingredient as a pharmaceutical composition. Accordingly, the invention further provides pharmaceutical compositions, which may be administered in the cancer treatment methods of the present invention. The pharmaceutical compositions include therapeutically effective amounts of a compound of formula (I) and salts or solvates thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of formula (I), depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The compound of formula (I) may be administered by any appropriate route. Suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination.

The method of the present invention may also be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged. Anti-neoplastic therapies are described for instance in International Application No. PCT US 02/01130, filed Jan. 14, 2002, which application is incorporated by reference to the extent that it discloses anti-neoplastic therapies. Combination therapies according to the present invention thus include the administration of at least one compound of formula (I) as well as optional use of other therapeutic agents including other anti-neoplastic agents. Such combination of agents may be administered together or separately and, when administered separately this may occur simultaneously or sequentially in any order, both close and remote in time. The amounts of the compound of formula (I) and the other pharmaceutically active agent(s) and the relative timings of administration will be selected in order to achieve the desired combined therapeutic effect.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The agents for use according to the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Agents for use according to the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists that may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

As indicated, therapeutically effective amounts of a specific compound of formula (I) is administered to a mammal. Typically, the therapeutically effective amount of one of the administered agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

Typically, the compound of formula (I) will be given in the range of 0.1 to 100 mg/kg body weight of recipient (mammal) per day and more usually in the range of 1 to 10 mg/kg body weight per day.

As recited above the present invention is directed to cancer treatment methods which includes administration of N-{3-chloro-4-[(3-fluorobenzyl)oxy]phenyl}-6-[5-({[2-(methanesulphonyl)ethyl]amino}methyl)-2-furyl]-4-quinazolinamine (GW572016) as well as salts and/or solvates thereof.

Bladder cancer is the 5^{th} most common malignancy in Europe and the 4^{th} most common malignancy in the United States (Jensen O, Esteve J. Eur J Cancer 1990; 26:1167-256; Jemal A, Thomas A. Cancer Statistics, 2002. C A Cancer J Clin 2002; 52:23-47). World-wide, the vast majority of bladder cancer is of the transitional cell type (TCC), which comprise 90 to 95% of urothelial tumours. These tumours may occur anywhere along the urinary tract, including the renal pelvis, ureters, bladder (90%), and proximal two-thirds of the urethra. Of the patients who present with clinically localised, muscle-invasive TCC, 20 to 80% can be cured with adequate local treatment consisting of either surgery or radiotherapy. Other patients will develop either local relapse or metastatic disease with little chance of cure. (de Mulder p, van der Meijden A. Bladder cancer. In: Oxford Textbook of Oncolog. 2nd ed. Oxford, N.Y.: Oxford University Press; 2002).

Many cytotoxic agents have been evaluated in the treatment of urothelial tumors including MVAC (methotrexate, vinblastine, doxorubicin and cisplatin) and CMV which are currently regarded as the gold standard for 1^{st} line treatment, in the United States and Europe respectively (Loehrer P, Einhorn L. J Clin Oncol 1992; 10:1066-1073); von der Maase H, Hansen S., J Clin Oncol 2000; 18:3068-3077). Gemcitabine/cisplatin (GC) is an alternative regimen to MVAC that has better safety profile and is being increasingly used, especially since gemcitabine is approved in 1^{st} line setting.

Second line treatment of locally advanced or metastatic TCC of the urothelial tract is less well defined. There are no approved therapies for patients with relapsed bladder and/or other urothelial cancers whose disease progresses following platinum-based chemotherapy. The most commonly used agents are taxanes, gemcitabine, mitomycin, and anthracyclines. Response rates vary between 7% and 27%, and time to progression (TTP) and overall survival (OS) are usually short. Because of the poor prognosis of bladder cancer and the inadequacy of the available therapies, this setting therefore represents a true unmet medical need, and mandates further investigation for effective therapies.

Overexpression of EGFR (erbB1) and erbB2 in bladder cancer has been correlated with advanced stage and high grade of the tumour (Mellon J, Lunec J., J Uro 1996; 155:321-6; Orlando C, Sestini R., J Urol 1996; 156:2089-2093). ErbB1 and erbB2 are expressed in 72.2% and 44.5% of primary bladder cancer samples, respectively. Combined expression of erbB1 and erbB2 is found in 33.9% of samples (Chow N-H, Chan S-H., Clin Cancer Res 2001; 7:1957-1962). GW572016, as a potent and selective dual inhibitor of both erbB1 and erbB2, may therefore provide an effective, well-tolerated and convenient treatment option for patients with locally advanced or metastatic TCC of the urothelial tract.

In one embodiment of the present invention, there is provided a method of treating urothelial cancer in a mammal, comprising: administering to said mammal therapeutically effective amounts of a compound of formula (I) and salts or solvates thereof. In another embodiment, the compound is a compound of formula (I′), preferably the compound of formula (I″). In one embodiment of the present invention, there is provided a method of treating bladder cancer in a mammal, comprising: administering to said mammal therapeutically effective amounts of a compound of formula (I) and salts or solvates thereof. In another embodiment, the compound is a compound of formula (I′), preferably the compound of formula (I″). In an additional embodiment of the present invention, there is provided a method of treating advanced or metastatic bladder cancer in a mammal, comprising: administering to said mammal therapeutically effective amounts of a compound of formula (I) and salts or solvates thereof. In a further embodiment of the present invention, there is provided a method of treating transition cell bladder cancer in a mammal, comprising: administering to said mammal therapeutically effective amounts of a compound of formula (I) and salts or solvates thereof.

Pancreatic cancer is one of the top 10 causes of death from cancer in the Western world. Surgical resection improves the outlook, although only about 10 percent of patients with pancreatic cancer are eligible for the procedure. Most treatment failures are due to local recurrence, hepatic metastases, or both and occur within one to two years after surgery. Advanced pancreatic cancer has few therapeutic options and a dismal prognosis. Less than 2% of patients with pancreatic cancer survive 5 years. Patients presenting with locally advanced pancreatic cancer are either treated with chemoradiotherapy, generally a fluorouracil (FU)- or gemcitabine-based regimen, or with gemcitabine alone. For tumors with distant metastases, gemcitabine has become the standard of care after a small randomized trial showed a statistically significant improvement in cancer-related symptoms (23.8% v 4.8% clinical benefit response) and a modest improvement in overall survival (5.6 v 4.4 months) compared with a regimen that was FU-based. Recent attempts in randomized controlled trials to improve overall survival by either combining gemcitabine with FU or by replacing gemcitabine by BAY12-9566, marimastat, or the farnesyltransferase inhibitor tipifarnib, did not improve the survival of gemcitabine-treated pancreatic cancer patients. ErbB1 expression is about 30% and erbB2 expression is about 70%.

In one embodiment of the present invention, there is provided a method of treating pancreatic cancer in a mammal, comprising: administering to said mammal therapeutically effective amounts of a compound of formula (I) and salts or solvates thereof. In another embodiment, the compound is a compound of formula (I′), preferably the compound of formula (I″).

Estimates of prostate-cancer incidence indicate that there will be over 220,000 new cases diagnosed in the US in 2003, resulting in over 28,000 deaths. In the United Kingdom, Italy, Spain, France, and Germany, over 110,000 cases of prostate cancer were diagnosed and over 46,000 deaths occurred due to this disease. In addition, prostate cancer is the fourth most frequent cancer of men worldwide. Nearly half of the patients with prostate cancer recur with advanced disease after definitive local therapy consisting of radiation or prostatectomy. Although patients with advanced prostate cancer are effectively treated with androgen ablation, the effect on disease progression is temporary. These patients ultimately become unresponsive to androgen ablation and are then classified as having hormone-refractory prostate cancer (HRPC) [Crawford, 1989; Eisenberger, 1994]. Standard options for patients with HRPC include secondary hormonal therapies or chemotherapy. Combination chemotherapy regimens involving agents that affect microtubule integrity appear to have activity with tolerable adverse effects. However, despite high prostate specific antigen (PSA) response rates, median duration of response is limited to approximately 6 months. Survival benefits have yet to be demonstrated. Although advances in palliation of symptoms and improvements in quality of life have been obtained with chemotherapy and steroids, innovative approaches are needed to improve survival rates. Novel approaches to the treatment of prostate cancer include the use of targeted therapies to pathways critical to tumor cell survival. For treatment of metastatic HRPC, Mitoxantrone/prednisone or estramustine are commonly used. In addition, docetaxel (75 mg/m2, every 3 weeks) was approved May 19, 2004 in combination with prednisone as first-line therapy for metastatic HRPC. Currently, multiple LHRH agonists and anti-androgens are used in hormone sensitive prostate cancer. Several small trials with chemotherapy agents are underway. The ability to delay the use of androgen blockade and the toxicity associated with this therapy could be a clinically important advantage of erbB1/2 inhibitors in this setting. Both erbB1 and erbB2 are expressed in prostate cancer. The majority of studies have indicated increased expression with progression of prostate cancer as it becomes refractory to standard hormone therapy (41-100% erbB1; 20-80% erbB2). Clinical studies with either ErbB1 or ErbB2 targeted therapies have not been promising in prostate cancer. As expression of both erbB1 and erbB2 correlate with the progression of prostate cancer to hormone-refractory disease, a dual inhibitor of the erbB receptors may provide a more effective treatment option than a drug that specifically inhibits either receptor alone.

In one embodiment of the present invention, there is provided a method of treating prostate cancer in a mammal, comprising: administering to said mammal therapeutically effective amounts of a compound of formula (I) and salts or solvates thereof. In another embodiment, the compound is a compound of formula (I′), preferably the compound of formula (I″). In one embodiment of the present invention, there is provided a method of treating hormone refractory prostate cancer in a mammal, comprising: administering to said mammal therapeutically effective amounts of a compound of formula (I) and salts or solvates thereof. In another embodiment, the compound is a compound of formula (I′), preferably the compound of formula (I″).

Colorectal cancer is the fourth most common type of cancer in Western society and the second leading cause of cancer-related death in North America. The annual incidence of colorectal cancer in the United States is approximately 148,300 (affecting 72,600 males and 75,700 females), with 56,600 deaths (in 27,800 males and 28,800 females). The lifetime risk of colorectal cancer in the general population is about 5 to 6 percent. Patients with a familial risk—those who have two or more first- or second-degree relatives (or both) with colorectal cancer—make up approximately 20 percent of all patients with colorectal cancer, whereas approximately 5 to 10 percent of the total annual burden of colorectal cancer is inherited in an autosomal dominant manner.

Although surgical resection alone is potentially curative, local or distant recurrences develop in many patients, and those with the highest risk of recurrence are advised to receive fluorouracil-based systemic adjuvant chemotherapy, which has been shown to be beneficial in a number of cooperative-group trials and analyses.

ErbB2 is moderately to highly expressed in primary tumors. In vitro expression of erbB2 is associated with progression in the adenoma-to-carcinoma sequence, tumor stage, invasive potential, and is an independent prognostic factor for survival. ErbB1 expression is lower than erbB2 (40% versus 60%) although its role in growth may be more potent than erbB2. Co-expression of erbB1 and erbB2 and erbB3 is ˜30% (all grades).

In one embodiment of the present invention, there is provided a method of treating colorectal cancer in a mammal, comprising: administering to said mammal therapeutically effective amounts of a compound of formula (I) and salts or solvates thereof. In another embodiment, the compound is a compound of formula (I′), preferably the compound of formula (I″). In one embodiment, the cancer is colon cancer. In another embodiment, the cancer is rectal cancer.

The annual incidence of lung cancer worldwide is 1.24 million people. In addition, 1.1 million people globally are estimated to die from lung cancer every year [Parkin, 2001]. Approximately three-quarters of these patients will have non-small cell lung cancer (NSCLC), and most will have locally advanced or metastatic disease at diagnosis. Currently, there is no curative therapy for these patients; therefore, the goal of chemotherapy in NSCLC patients is to extend the quality of life through palliation of symptoms. First-line combination chemotherapy of advanced NSCLC can produce response rates of 20-50% with additional patients achieving stabilization of disease. Current standard first-line therapy for NSCLC includes cisplatin combined with vinorelbine or gemcitabine or carboplatin combined with paclitaxel or docetaxel [Schiller, 2002; Fossella, 2003]. The time to progression (TTP) and overall survival (OS) in the four platinum doublets reported by Schiller et al, were similar, 4 months and 8 months respectively. Fossella et al., reported a TTP and OS of 5 months and 9 months with the combination of carboplatin and docetaxel.

The use of second-line chemotherapy should achieve a balance between a desired benefit (either prolongation of survival or improvement in the quality of life) and drug-related toxicity. Docetaxel, vinorelbine, gemcitabine, irinotecan, paclitaxel, and premetrexed have shown activity as second-line treatment in patients with NSCLC. Currently, only docetaxel is indicated for the treatment of patients with locally advanced or metastatic non-small cell lung cancer after failure of prior platinum-based chemotherapy. EGFR inhibitors have also been looked at. Gefitinib (Iressa®) monotherapy has been approved for use in the US for patients with locally advanced or metastatic NSCLC after failure of both platinum-based and docetaxel chemotherapies. Preliminary results from a phase III study of erlotinib (Tarceva®) in relapsed NSCLC patients indicated improvement of overall survival rates was achieved. Cetuximab (Erbitux®), a monoclonal antibody that targets erbB1, was recently approved in CRC and ongoing trials are exploring its activity in NSCLC.

In one embodiment of the present invention, there is provided a method of treating a lung cancer in a mammal, comprising: administering to said mammal therapeutically effective amounts of a compound of formula (I) and salts or solvates thereof. In another embodiment, the compound is a compound of formula (I′), preferably the compound of formula (I″). In one embodiment, the cancer is non small cell lung cancer.

Approximately 14,300 women die from ovarian cancer in the US per year. It is the fifth most common cancer in women and the leading cause of death from genital cancers. The 5-year survival rate for ovarian cancer is approximately 95% for women with localized disease. However, approximately 60% of women have advanced stage (stage III/IV) ovarian cancer at diagnosis, and the 5-year survival rate with distant disease is only 31%. Age and race influence the incidence of ovarian cancer and the survival rate. Women age 65 years and older have a higher incidence of disease (56.3 of. 100,000 compared with 11.2 of 100,000 for women younger than 65 years) and a significantly poorer 5-year survival (32.9% compared with 65.8%).

In the US, standard therapy for women with advanced epithelial ovarian cancer has developed from a series of randomized trials performed primarily by the Gynecologic Oncology Group (GOG). In 1996, this group reported the results of a randomized comparison of cisplatin and cyclophosphamide versus cisplatin and paclitaxel in patients with previously untreated advanced stage III and IV disease. The cisplatin plus paclitaxel regimen was judged superior on the basis of the following results of that trial: an overall improved response rate (73% v 60%; P=0.01); an increased clinical complete response rate (54% v 32%); an increase in progression-free survival (PFS; 18.1 v 13.6 months; P<0.001); and, most importantly, an increased overall median survival (38 v 24 months; P<0.001). The results of this study were subsequently confirmed by a European-Canadian trial in patients with stage IIB through IV epithelial ovarian cancer who were similarly randomly assigned to a cisplatin plus cyclophosphamide regimen versus cisplatin plus paclitaxel. In the latter study, cisplatin was combined with paclitaxel administered as a 3-hour infusion, whereas in the GOG trial, paclitaxel was administered as a 24-hour infusion. Despite these differences in protocol design, both studies demonstrated superiority of initial treatment with cisplatin plus paclitaxel in patients with previously untreated advanced ovarian cancer. Carboplatin, an analog of cisplatin, has less nonhematologic toxicity than the parent compound cisplatin and the combination of carboplatin plus paclitaxel has also been found to be an active regimen.

ErbB1 is commonly expressed on tumor samples, while erbB-2 is less so. Co-expression of erbB1 and erbB2 is also common. In xenograft animal models and patient samples, erbB1 is an independent prognostic marker for poor survival and chemotherapy resistance. erbB1 overexpression is about 70%, while erbB2 over-expression is about 30-60%. Co-expression of erbB1 and erbB2 is also common: about 30-60%. ErbB1 expression is an independent prognostic factor for survival and DFS in multivariate analysis, however erbB2 prognostic value is rather weak. In ovarian cancer cell lines, there is evidence for erbB1 growth-dependency.

In one embodiment of the present invention, there is provided a method of treating an ovarian cancer in a mammal, comprising: administering to said mammal therapeutically effective amounts of a compound of formula (I) and salts or solvates thereof. In another embodiment, the compound is a compound of formula (I′), preferably the compound of formula (I″).

Vulval cancer is a rare cancer in women which affects the vulva, that is the external female sex organs and is more common in women middle aged and older. Standard therapy for vulval cancer involves surgery, radiotherapy, and/or chemotherapy, typically topical administration of fluorouracil in early stage disease and administration of cisplatin as a montherapy or with other agents, for late or metastatic disease.

In one embodiment of the present invention, there is provided a method of treating vulval cancer in a mammal, comprising: administering to said mammal therapeutically effective amounts of a compound of formula (I) and salts or solvates thereof. In another embodiment, the compound is a compound of formula (I′), preferably the compound of formula (I″).

Cancer of the cervix is the second most common cancer in women worldwide and is a leading cause of cancer-related death in women in underdeveloped countries. Worldwide, approximately 500,000 cases of cervical cancer are diagnosed each year. Routine screening has decreased the incidence of invasive cervical cancer in the United States, where approximately 13,000 cases of invasive cervical cancer and 50,000 cases of cervical carcinoma in situ (i.e., localized cancer) are diagnosed yearly. Invasive cervical cancer is more common in women middle aged and older and in women of poor socioeconomic status, who are less likely to receive regular screening and early treatment. There is also a higher rate of incidence among African American, Hispanic, and Native American women. Standard therapy for cervical cancer involves surgery, radiotherapy, and/or chemotherapy, typically administration of cisplatin as a montherapy or with other agents, such as fluorouracil, is indicated.

In one embodiment of the present invention, there is provided a method of treating a cervical cancer in a mammal, comprising: administering to said mammal therapeutically effective amounts of a compound of formula (I) and salts or solvates thereof. In another embodiment, the compound is a compound of formula (I′), preferably the compound of formula (I″).

Involvement of erbB2 in endometrial cancer is through over expression, with or without gene amplification. Ten (10) percent to 15% of endometrial cancers display overexpression of erbB2 compared with normal endometrial epithelium, as quantitated by immunohistochemistry. Some studies have documented erbB2 gene amplification in endometrial cancers in a subset of tumors that show overexpression. Overexpression if erbB2 seems to be confined to a subset of high-grade and/or advanced stage tumors. Correlation of erbB2 expression with clinical outcome is less conclusive, although a trend has been observed correlating overexpression and worsening prognosis. (Am J Obstet Gynecol 164:15-21, 1991; Gynecol Oncol 47:179-185, 1992; Gynecol Oncol 53:84-92, 1994.)

In one embodiment of the present invention, there is provided a method of treating an endometrial cancer in a mammal, comprising: administering to said mammal therapeutically effective amounts of a compound of formula (I) and salts or solvates thereof. In another embodiment, the compound is a compound of formula (I′), preferably the compound of formula (I″).

Renal cell carcinoma (RCC) is the 6^{th} leading cause of cancer death in the US, accounting for 3% of adult malignancies. There were about 31,200 cases diagnosed in 2000, with approximately 12,000 deaths in the US. In the rest of the world, rates vary more than 10-fold. Incidence is highest in Scandinavia, and other parts of Northern Europe, but lower in England and Wales. The lowest rates are reported in India, Chinese and Japanese population, and areas of Central and South America. Incidence has been steadily rising during the past 25 years. ErbB1 over-expression ranges from 40% to over 80%. Over-expression correlates with tumor stage and survival. The role of cytokine therapy with IL-2 and/or IFN-alpha in prolonging survival for patients with advanced and metastatic RCC remains controversial, with a response rate of about 15% and substantial toxicity from therapy. The response duration ranged from 6-10 months. RCC is inherently resistant to chemotherapy and hormonal therapy, since no agent consistently achieves a response in more than 10% of patients.

In one embodiment of the present invention, there is provided a method of treating renal cancer in a mammal, comprising: administering to said mammal therapeutically effective amounts of a compound of formula (I) and salts or solvates thereof. In another embodiment, the compound is a compound of formula (I′), preferably the compound of formula (I″). In one embodiment, the renal cell cancer is renal cell carcinoma.

Mesothelioma is a cancer of the pleural or peritoneal lining. Over-expression of the epidermal growth factor receptor (EGFR) is a common finding in many solid tumors, including lung, breast and mesothelioma. The overexpression has been shown to correlate with both a poor prognosis and resistance to radiation and chemotherapy. Recent evidence suggests that up-regulation and activation of EGFR may play a critical role in early carcinogenic events. Carcinogenic asbestos fibers, known to be a cause of mesothelioma, are known to upregulate the expression of the EGFR. Exposure of MET 5A cells to asbestos leads to the activation of nuclear factor-kB (NF-kB), a transcription factor important in the regulation of a number of genes intrinsic to inflammation, proliferation and lung defences. Modulation of the asbestos-mediated EGFR/NF-kB signalling pathway may be important in the development of novel therapeutic strategies for both the chemoprevention and treatment of malignant mesothelioma (Faux, EGFR Induced Activation of NF-kB in Mesothelial Cells by Asbestos Is Important in Cell Survival, Proceedings of the American Association for Cancer Research, AACR, Vol. 42, March 2001).

In one embodiment of the present invention, there is provided a method of treating mesothelioma in a mammal, comprising: administering to said mammal therapeutically effective amounts of a compound of formula (I) and salts or solvates thereof. In another embodiment, the compound is a compound of formula (I′), preferably the compound of formula (I″).

Cancers arising from the esophagus, including the gastroesophageal junction, are relatively uncommon in the United States, with 13,900 new cases and 13,000 deaths reported in 2003. The risk increases with age, with a mean age at diagnosis of 67 years. Esophageal cancer is the seventh leading cause of death from cancer among American men. Worldwide, esophageal cancer is the sixth leading cause of death from cancer. More than 90 percent of esophageal cancers are either squamous-cell carcinomas or adenocarcinomas. Approximately three quarters of all adenocarcinomas are found in the distal esophagus, whereas squamous-cell carcinomas are more evenly distributed between the middle and lower third. Overall, more than 50 percent of patients have unresectable or metastatic disease at the time of presentation. The overall survival rate at five years is poor and currently is 14 percent. Although both squamous-cell carcinoma and adenocarcinoma of the esophagus are responsive to chemotherapy, no regimens are currently approved. Expression of ErbB1 (30% adenocarcinoma; 70% squamous) and appears to be a prognostic indicator in patients with esophageal cancer. ErbB2 expression is about 25% in adenocarcinomas of the distal esophagus and GE junction.

In one embodiment of the present invention, there is provided a method of treating esophageal cancer in a mammal, comprising: administering to said mammal therapeutically effective amounts of a compound of formula (I) and salts or solvates thereof. In another embodiment, the compound is a compound of formula (I′), preferably the compound of formula (I″).

Several clinicopathological studies have examined the overexpression of EGFR and erbB2 in malignant salivary gland tumors using immunohistochemistry. For adenoid cyctic carcinoma (ACC), the frequency of EGFR overexpression varies from 0-85%, and likewise the frequency of erbB2 overexpression varies widely from 0-100%. These frequencies were generally based on small series with limited sample sizes. Other factors such as differences in laboratory staining and scoring methods, and choices of antibodies, likely contributed to the heterogeneity of the results. In a recently published large series of 137 salivary gland carcinomas (Glisson et al., ClinCanRes, 10:944-46, 2004), the overall frequency of erbB2 overexpression (2+ to 3+ complete membrane staining in at least 10% of tumor cells were scored as positive for overexpression) was 17% (23 of 137) among all histological subtypes of salivary gland carcinomas. Overexpression of erbB2 was found to be rare in ACC (4%, 3 of 70) whereas overexpression was common in salivary duct cancers (83%, 10 of 12). This observation was felt to be consistent with the typical high-grade histological features and aggressive behavior of the salivary duct subtype as well as its histogenetic similarity to breast cancer.

In one embodiment of the present invention, there is provided a method of treating salivary gland cancer in a mammal, comprising: administering to said mammal therapeutically effective amounts of a compound of formula (I) and salts or solvates thereof. In another embodiment, the compound is a compound of formula (I′), preferably the compound of formula (I″).

Hepatocellular carcinoma (HCC) is one of the most common cancers worldwide with 1.2 million cases annually. Its incidence is rapidly increasing due to widespread hepatitis C infection. There were approximately 17,300 cases in the US in 2003 with 14,400 deaths. Surgery is the treatment of choice for the minority of patients with resectable disease. Therapy for localized HCC includes surgery, radiofrequency ablation, ethanol injection, or trans-arterial chemoembolization. There is no established adjuvant therapy and the majority of tumors recur. The prognosis for recurrent or metastatic HCC is poor with median survival of 6 months. Doxorubicin as a single agent or in combination is a common treatment for advanced HCC; however, the response rate is low and there is no therapy that improves survival in this setting. Chemoembolism or transarterial embolization with chemotherapy are also commonly performed. Overexpression of erbB1 has been reported in HCC with varying degress of overexpression reported. ErbB1 expression in tumors is correlated with aggressive growth, poor prognosis and resistance to chemotherapy. ErbB1 and erbB2 expression in hepatoma cell lines are important regulators of growth.

In one embodiment of the present invention, there is provided a method of treating a hepatocellular cancer in a mammal, comprising: administering to said mammal therapeutically effective amounts of a compound of formula (I) and salts or solvates thereof. In another embodiment, the compound is a compound of formula (I′), preferably the compound of formula (I″).

EGFR is frequently overexpressed In malignant tumors of the brain. (Heimberger et al, ClinCanRes, 8:3496-3502, 2002), including gliomas (Bredel et al, ClinCanRes, 5:1786-1792, 1999). Accordingly, GW572016, as a potent and selective dual inhibitor of both erbB1 and erbB2, may therefore provide an effective, well-tolerated and convenient treatment option for patients with brain cancer, including gliomas.

In one embodiment of the present invention, there is provided a method of treating brain cancer in a mammal, comprising: administering to said mammal therapeutically effective amounts of a compound of formula (I) and salts or solvates thereof. In another embodiment, the compound is a compound of formula (I′), preferably the compound of formula (I″). In one embodiment, the brain cancer is a glioma.

Melanoma is a very serious form of skin cancer. It begins in melanocytes—cells that make the skin pigment called melanin. Although melanoma accounts for only about 4% of all skin cancer cases, it causes most skin cancer-related deaths. Typically, melanoma is treated with surgery and in high risk cases is followed by immunotherapy with, for instance interferon α-2b.

In one embodiment of the present invention, there is provided a method of treating melanoma in a mammal, comprising: administering to said mammal therapeutically effective amounts of a compound of formula (I) and salts or solvates thereof. In another embodiment, the compound is a compound of formula (I′), preferably the compound of formula (I″).

In the foregoing cancer treatment methods of the present invention the compounds of formulae (I), (I′), and (I″) are as described above.

The following examples are intended for illustration only and are not intended to limit the scope of the invention in any way.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

g (grams); mg (milligrams);

L (liters); mL (milliliters);

μL (microliters); psi (pounds per square inch);

M (molar); mM (millimolar);

N (Normal) Kg (kilogram)

i. v. (intravenous); Hz (Hertz);

MHz (megahertz); mol (moles);

mmol (millimoles); RT (room temperature);

min (minutes); h (hours);

mp (melting point); TLC (thin layer chromatography);

Tᵣ (retention time); RP (reverse phase);

DCM (dichloromethane); DCE (dichloroethane);

DMF (N,N-dimethylformamide); HOAc (acetic acid);

TMSE (2-(trimethylsilyl)ethyl); TMS (trimethylsilyl);

TIPS (triisopropylsilyl); TBS (t-butyldimethylsilyl);

HPLC (high pressure liquid chromatography);

THF (tetrahydrofuran); DMSO (dimethylsulfoxide);

EtOAc (ethyl acetate); DME (1,2-dimethoxyethane);

EDTA ethylenediaminetetraacetic acid

FBS fetal bovine serum

IMDM Iscove's Modified Dulbecco's medium

IMS Industrial Methylated Spirits

PBS phosphate buffered saline

RPMI Roswell Park Memorial Institute

RIPA buffer *

RT room temperature

*150 mM NaCl, 50 mM Tris-HCl, pH 7.5, 0.25% (w/v)-deoxycholate, 1% NP-40, 5 mM sodium orthovanadate, 2 mM sodium fluoride, and a protease inhibitor cocktail.

Unless otherwise indicated, all temperatures are expressed in ° C. (degrees Centigrade). All reactions conducted under an inert atmosphere at room temperature unless otherwise noted.

GW572016F is lapatanib whose chemical name is N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate.

### Example 1

### Preparation of GW572016F

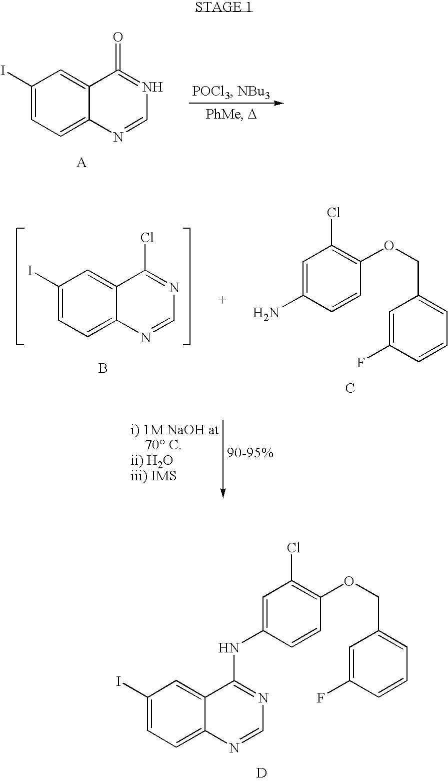

A stirred suspension of 3H-6-Iodoquinazolin-4-one (compound A) in toluene (5 vols) was treated with tri-n-butylamine (1.2 eq.) at 20 to 25° C., then heated to 90° C. Phosphorous oxychloride (1.1 eq) was added, the reaction mixture was then heated to reflux. The reaction mixture was cooled to 50° C. and toluene (5 vols) added. Compound C (1.03 eq.) was added as a solid and the slurry was warmed back to 90° C. and stirred for 1 hour. The slurry was transferred to a second vessel; the first vessel was rinsed with toluene (2 vol) and combined with the reaction mixture. The reaction mixture was cooled to 70° C. and 1.0 M aqueous sodium hydroxide solution (16 vols) added dropwise over 1 hour to the stirred slurry maintaining the contents temperature between 68-72° C. The mixture was stirred at 65-70° C. for 1 hour and then cooled to 20° C. over 1 hour. The suspension was stirred at 20° C. for 2 hours, the product collected by filtration, and washed successively with water (3×5 vols) and ethanol (IMS, 2×5 vols), then dried in vacuo at 50-60° C. Volumes are quoted with respect of the quantity of Compound A used. Percent yield range observed: 90 to 95% as white or yellow crystals.

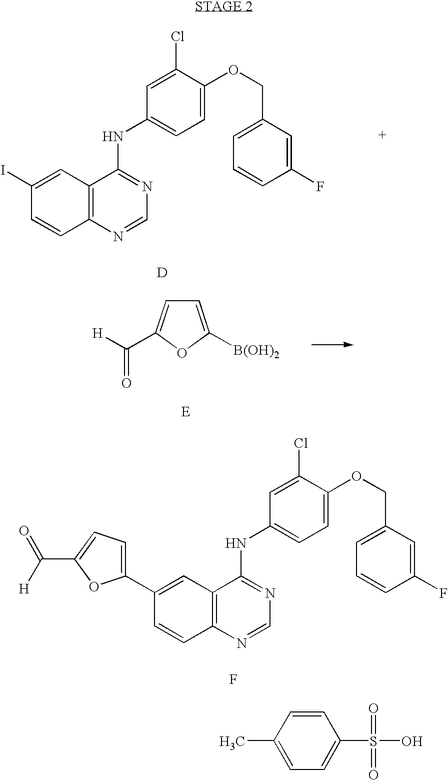

A mixture of N-{3-chloro-4-[(3-fluorobenzyl)oxy]phenyl}-6-iodo-4-quinazolinamine—compound D (1 wt), boronic acid—compound E (0.37 wt, 1.35 equiv), and 10% palladium on charcoal (0.028 wt, 50% water wet) was slurried in IMS (15 vol). The resultant suspension was stirred for 5 minutes, treated with di-isopropylethylamine (0.39 vol, 1.15 equiv) and then heated to ca 70° C. for ca 3 hours when the reaction was complete (determined by HPLC analysis). The mixture was diluted with tetrahydrofuran (THF, 15 vol) and then hot-filtered to remove the catalyst. The vessel was rinsed with IMS (2 vol).

A solution of p-toluenesulfonic acid monohydrate (1.5 wt, 4 equiv) in water (1.5 vol) was added over 5-10 minutes to the filtered solution maintained at 65° C. After crystallisation the suspension was stirred at 60°-65° C. for 1 hour, cooled to ca 25° C. over 1 hour and stirred at this temperature for a further 2 hours. The solid was collected by filtration, washed with IMS (3 vol) then dried in vacuo at ca 50° C. to give the desired compound F as a yellow-orange crystalline solid (isolated as the ethanol solvate containing approximately 5% w/w EtOH).

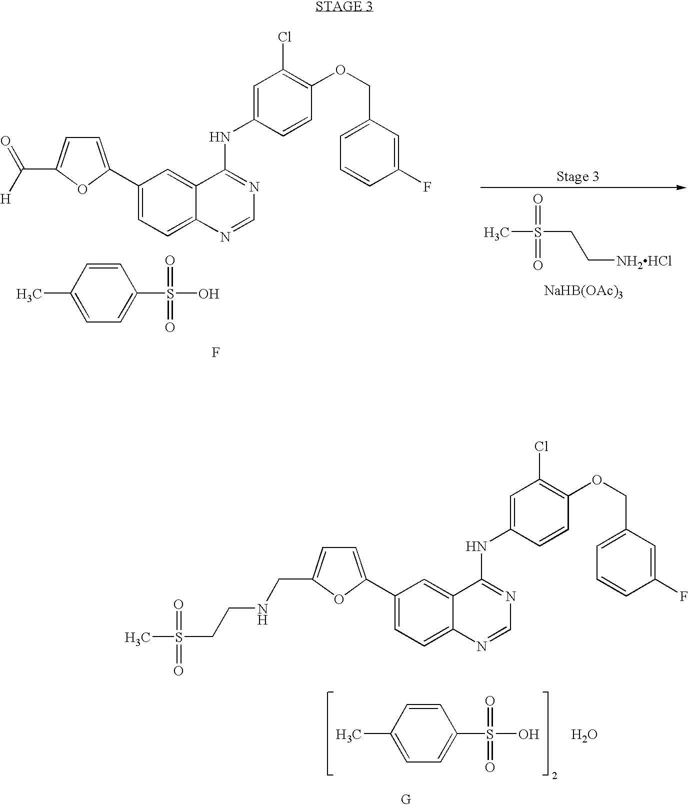

Compound F^{#} (1 wt) and 2-(methylsulfonyl)ethylamine hydrochloride (0.4 wt, 1.62 equiv.) were suspended in THF (10 vols). Sequentially, acetic acid (0.354 vol., 4 equiv.) and di-isopropylethylamine (DIPEA, 1.08 vol., 4.01 equiv.) were added. The resulting solution was stirred at 30°-35° C. for ca 1 hour then cooled to ca 22° C. Sodium tri-acetoxyborohydride (0.66 wt, 2.01 equiv.) was then added as a continual charge over approximately 15 minutes (some effervescence is seen at this point). The resulting mixture was stirred at ca 22° C. for ca 2 hours then sampled for HPLC analysis. The reaction was quenched by addition of aqueous sodium hydroxide (25% w/w, 3 vols.) followed by water (2 vols.) and stirred for ca 30 minutes (some effervescence was seen at the start of the caustic addition).

The aqueous phase was then separated, extracted with THF (2 vols) and the combined THF extracts were then washed twice with 25% w/v aqueous ammonium chloride solution (2×5 vols)². A solution of p-toluenesulfonic acid monohydrate (p-TSA, 0.74 wt, 2.5 equiv.) in water (1 vol)¹ was prepared, warmed to ca 60° C., and GW572016F (Compound G) (0.002 wt) seeds were added.

The THF solution of the free base of GW572016 was added to the p-TSA solution over at least 30 minutes, while maintaining the batch temperature at 60±3° C. The resulting suspension was stirred at ca 60° C. for 1-2 hours, cooled to 20-25° C. over an hour and aged at this temperature for ca 1 hr. The solid was collected by filtration, washed with 95:5 THF:Water (3×2 vols) and dried in vacuo at ca 35° C. to give GW572016F—compound G as a bright yellow crystalline solid. Expected yield 80% theory, 117% w/w.

¹ Minimum reaction volume ca 1 vol.
² Maximum reaction volume ca 17 vol.
# Corrected for assay.

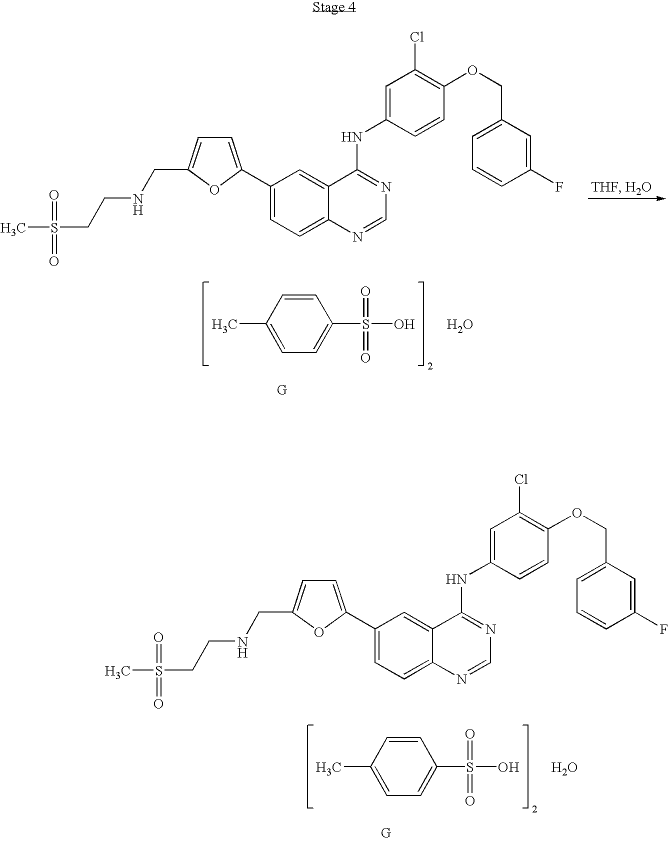

A suspension of the ditosylate monohydrate salt of N-{3-Chloro-4-[(3-fluorobenzyl)oxy]phenyl}-6-[5-({[2-(methane sulphonyl)ethyl]amino}methyl)-2-furyl]-4-quinazolinamine—compound G (1 wt), in tetrahydrofuran (THF, 14 vol) and water (6 vol) was heated to ca 55°-60° C. for 30 minutes to give a solution which was clarified by filtration and the lines washed into the crystallisation vessel with THF/Water (7:3 ratio, 2 vol). The resultant solution was heated to reflux and tetrahydrofuran (9 vol, 95% w/w azeotrope with water) was distilled off at atmospheric pressure.

The solution was seeded with N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate (0.002 wt). Once the crystallisation was established water (6 vol) was added while maintaining the reaction temperature above 55° C. The mixture was cooled to 5°-15° C. over ca 2 hours. The solid was collected by filtration, washed with tetrahydrofuran/water (3:7 ratio, 2 vol) then tetrahydrofuran/water (19:1 ratio, 2 vol) and dried in vacuo at 45° C. to give N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate as a bright yellow crystalline solid.

### Example 2

### Determination of Concentrations of GW 572016F that Inhibit 50% of Cell Growth (IC₅₀)

The human bladder cell lines, HT-1197, HT-1376 and T24, and the ovarian cell line, SKOV3, were obtained from the American Type Culture Collection. The cells were maintained in tissue culture flasks in RPMI 1640 (Invitrogen # 22400-089) with 10% fetal bovine serum (FBS, HyClone # SH30071.03) and were incubated at 37° Celsius in an atmosphere of 5% CO₂, until plating for IC₅₀ determination. For IC₅₀ determination, cells were plated in the appropriate medium at 5,000 cells per well in a 96-well tissue culture dish and returned to the incubator overnight. Approximately twenty-four hours after initial seeding, cells were exposed to the ditosylate salt form of GW 572016, GW 572016F. Cells were dosed in 50% RPMI and 50% low glucose DMEM medium containing, 5% FBS, 50 micrograms/ml gentamicin and 0.3% DMSO with three fold serial dilutions of GW 572016F. Final concentrations ranged from 30 micromolar to 0.00152 micromolar. After three days of compound exposure, the growth medium was removed by aspiration. Cell biomass was estimated by staining cells in 0.1 ml per well of methylene blue (Sigma #M9140, 0.5% in 50:50, ethanol:water), followed by incubation at room temperature for at least 30 minutes. Stain was aspirated and the plates rinsed by immersion in deionized water, followed by air drying. Stain was released from cells by the addition of 0.1 ml of solubilization solution (1.0% N-lauryl sarcosine, Sodium salt, Sigma #L5121 in PBS). Plates were incubated at room temperature for 40 minutes. Absorbance was read at 620 nM in a Tecan Spectra micro-plate reader. Percent inhibition of cell growth was calculated relative to untreated control wells. IC₅₀ values were interpolated using the method of Levenberg and Marquardt (Mager, 1972) and the equation: y=Vₘₐₓ*[1−(xⁿ/(Kⁿ+xⁿ))], where “K” is equal to IC₅₀.

Results are reported in Table 1 along with results for additional cell lines. The additional cell lines were prepared and exposed to GW572016F according to methods similar to those recited in Example 2. All cell lines are obtainable from the American Type Culture Collection and were plated at densities which provided logarithmic growth for the duration of compound exposure.

TABLE 1

| | | Growth |
|---|---|---|
| | | Inhibition |
| Tissue of Origin | Cell Line | (IC50 μM) |
| Bladder | H1197 | 7.78 |
| Bladder | H1376 | 3.34 |
| Bladder | T24 | 8.22 |
| Colon adenocarcinoma | SW480 | 6.86 |
| Colorectal carcinoma | Lovo | 3.39 |
| Colorectal carcinoma | HCT 116 | 5.87 |
| Colorectal - cecum | NCI-H747 | 1.49 |
| Colon adenocarcinoma | LS174T | 1.51 |
| Colon adenocarcinoma | DLD-1 | 3.63 |
| Colon adenocarcinoma | HT29 | 5.24 |
| Colon adenocarcinoma | SW620 | 6.75 |
| Colon adenocarcinoma | Colo 205 | 7.70 |
| Rectum adenocarcinoma | SW837 | 4.7 |
| Colorectal carcinoma | RKO | 5.48 |
| Pancreatic carcinoma | BxPC3 | 1.41 |
| Lung carcinoma | H157 | 5.98 |
| Lung carcinoid bronchus | NCI-H727 | 7.59 |
| Lung Carcinoma | NCI-H2009 | 11.5 |
| Lung Carcinoma | A549 | 4.98 |
| Lung Carcinoma | A427 | 5.95 |
| Lung Carcinoma | NCI-H460 | 9.00 |
| Lung Carcinoma | CaLu3 | 0.057 |
| NSCLC | NCI-H322 | 0.92 |
| NSCLC-alveolar | NCI-H358 | 0.27 |
| Lung epidermoid | Calu1 | 5.51 |
| Melanoma | SKMEL28 | 5.90 |
| Normal breast | HMEC | 1.34 |
| Normal Human Foreskin | HFF | 6.45 |
| Ovarian Carcinoma | SKOV3 | 1.25 |
| Prostate Carcinoma | PC3 | 7.15 |
| Prostate Carcinoma | LNCaP | 4.69 |
| Renal | 786O | 1.82 |
| Transfected erbB2 | H16 N2 | 0.03 |
| Vulval carcinoma | A431 | 0.23 |

### Example 3

### Clinical Study of Orally Administered Lapatinib as Single-Agent, Second-Line Treatment of Patients with Locally Advanced or Metastatic Transitional Cell Carcinoma of the Urothelial Tract

Fifty-eight patients with locally advanced or metastatic urothelial tumors, who had progressed after platinum-based therapy, received 1250 mg lapatinib once daily until disease progression or withdrawal. Safety and efficacy assessments (independent review) were carried out at 4 and 8-week intervals respectively. Patients were also assessed at withdrawal, and followed every 2 months until death. Data from 30 patients were reviewed at an interim analysis (16 weeks on study), and are presented herein.

The median age was 62 years. Most patients (67%) had visceral metastases. All patients had confirmed expression of erbB1 and/or erbB2 (1+, 2+ or 3+ by immunohistochemistry). Only 19 patients (63%) received lapatinib as intended second-line therapy. Three patients (10%) had tumour reduction that was qualified as partial responses (PR) at the initial assessment; however, only one PR was confirmed at 8 weeks. Eight patients (27%) had stable disease (SD), 5 with noted cytoreduction. Clinical benefit (≧6 months SD) was seen in 3 patients (10%). Five patients (17%) had progressive disease, 11 patients (37%) withdrew prior to week 8, and 3 patients (10%) were not evaluable. Disease progression was the most frequent reason for withdrawal.

Second-line treatment with oral lapatinib showed promising activity and was generally well tolerated in patients with locally advanced or metastatic urothelial tumors.

### Example 3A

### Clinical Phase II Study of Orally Administered Lapatinib as Single-Agent, Second-Line Treatment of Patients with Locally Advanced or Metastatic Transitional Cell Carcinoma of the Urothelial Tract

Methods: The primary endpoint was RECIST response rate (by independent radiologic review). Key eligibility criteria included stage 111B or 1V TCC of the bladder, progression following a first-line platinum-based regimen, measurable disease, expression of erbB1 and or erbB2 (1+, 2+ or 3+ by immunohistochemistry) and Karnofsky performance status of 70 or greater. Oral lapatinib (1250 mg, daily) was administered until disease progression or unacceptable toxicity. Tumor and safety assessments were performed every 8 and 4 weeks, respectively. Cardiac function was monitored at baseline and every 8 weeks. Tumor tissue was analyzed for a variety of biomarkers (TUNEL, p53, pAkt, Her3, pHer3, pErk, IGF-1R, Rb, pS6).

Results: Fifty-nine patients with locally advanced or metastatic TCC of the bladder were enrolled. Investigators reported 2 (3%) partial responses (PR) and 12 (20%) stable disease (SD); independent radiologic review reported 1 (2%) PR and 18 (31%) SD. Based upon investigator and independent review, six and three patients had durable SD lasting 4 and 6 months, respectively. At week 8, 10 patients had tumor growth up to 20%, 4 patients had cytostasis and 10 patients had cytoreduction. The majority of tumor shrinkage was short-lived; however, one patient remains on study for >56 weeks. The median TTP was 8.6 weeks (95% Cl, 8.00, 11.29) and median overall survival was 17.9 weeks (95% Cl, 13.14, 30.29). A trend towards increased clinical benefit was observed in patients with erbB1 or erbB2 2+ and 3+. Preliminary analysis suggests the following biomarkers may predict patients who will be refractory to lapatinib: high pHer3, high pErk and both mutant p53 & high pHer3. In contrast, patients with high pAkt and high IGF-1R were sensitive to lapatinib. Adverse events (AEs) with 10% frequency were diarrhea (39%), rash (32%), nausea (27%), vomiting (22%), asthenia (12%) and fatigue (10%). Grade 3/4 AEs occurring in more than one patient were vomiting (7%) and diarrhea (3%). One patient had an asymptomatic, Grade 2 decrease in cardiac ejection fraction.

Conclusions: In summary, lapatinib was well-tolerated and exhibits monotherapy activity in patients with relapsed, advanced or metastatic TCC of the bladder, demonstrated by clinical benefit (CR+PR+SD≧16 weeks) in 14% and 12% of patients as assessed by investigator and independent review, respectively. The median TTP of 8.6 weeks is comparable to reports of various chemotherapies in the second-line setting. There was a trend toward clinical benefit in patients with erbB1 or erbB2 2-3+ by immunohistochemistry.

### Example 4

### Clinical Study of Orally Administered Lapatinib in Patients with Solid Tumors

Eighty-one patients (pts) (27 colon, 7 lung, 6 adenocarcinoma of unknown primary (AUP), 5H&N, 6 renal, 6 breast, 4 ovarian, and 15 other (see Table II) were treated with lapatinib once (qd) or twice (bid) daily in a dose escalation scheme. Forty pts were administered 175-1800 mg qd and forty-one pts administered either 500, 750, or 900 mg bid. Pts were evaluated monthly and treated until disease progression or intolerable side effects. Clinical response was determined every 8 wks.

One CR (sustained for 16+ mos) was observed in an erbB1 overexpressing head and neck squamous cell carcinoma. Twenty-two pts, with various tumors most overexpressing either erbB1 or erbB2, experienced SD with a median duration of 4 mos (range 1-13+ months). Patients continuing therapy for >4 mos were administered lapatinib at doses≧1200 mg/day. Of the 22 SD pts, 2 with non-small cell lung cancer and AUP metastatic to lung, both progressing on previous therapy, remained on lapatinib for 12+ and 8+ mos, respectively. There were no cases of interstitial pneumonitis.

QD administration of lapatinib was well tolerated with evidence of clinical activity in this heavily pre-treated population. Results are illustrated in Table II. Only patients receiving lapatinib for more than 4 months are included in Table 2.

TABLE 2

| | # | Clinical |
|---|---|---|
| Tumor Type | Patients | Response |
| Colorectal | 27 | SD = 3 |
| Breast | 6 | SD = 2 |
| AUP | 6 | SD = 1 |
| NSCLC | 7 | SD = 3 |
| Renal | 6 | SD = 2 |
| Head and Neck | 9 | CR = 1; SD = 3 |
| Mesothelioma | 3 | SD = 1 |
| Ovarian | 4 | |
| Pancreatic | 2 | |
| Cervical | 2 | SD = 1 |
| Gastric | 1 | |
| Gatrointestinal | 1 | |
| Bronchoalveolar | 1 | |
| Prostate | 1 | |
| Esophogeal | 1 | |
| Neuroendocrine | 1 | |
| High Grade Sarcoma | 1 | |
| Myxoid liposarcoma | 1 | |
| Ocular melanoma | 1 | |

CR—complete response defined as disappearance of target lesions
PR—partial response defined as reduction of at least 30% in target lesions
SD—stable disease defined as no growth or some reduction in target lesion

What is claimed is:

1. A method of treating an EGFR and/or erbB2 overexpressing cancer in a mammal, comprising: administering to said mammal therapeutically effective amounts of a compound of formula (I″)

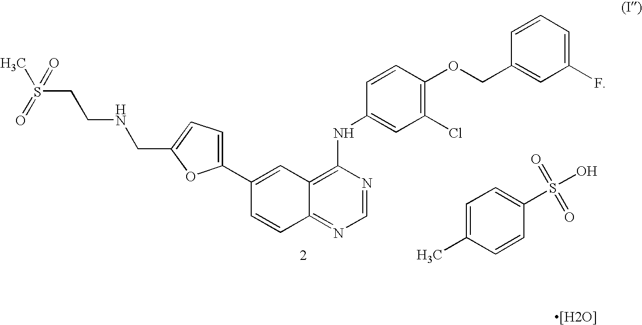

2. The method of claim 1, wherein the cancer is pancreatic cancer.

3. The method of claim 1, wherein the cancer is prostate cancer.

4. The method of claim 1, wherein the cancer is hormone refractory prostate cancer.

5. The method of claim 1, wherein the cancer is colorectal cancer.

6. The method of claim 1, wherein the cancer is colon cancer.

7. The method of claim 1, wherein the cancer is rectal cancer.

8. The method of claim 1, wherein the cancer is non small cell lung cancer.

9. The method of claim 1, wherein the cancer is ovarian cancer.

10. The method of claim 1, wherein the cancer is vulval cancer.

11. The method of claim 1, wherein the cancer is cervical cancer.

12. The method of claim 1, wherein the cancer is endometrial cancer.

13. The method of claim 1, wherein the cancer is mesothelioma.

14. The method of claim 1, wherein the cancer is esophageal cancer.

15. The method of claim 1, wherein the cancer is salivary gland cancer.

16. The method of claim 1, wherein the cancer is hepatocellular cancer.

17. The method of claim 1, wherein the cancer is brain cancer.

18. The method of claim 1, wherein the cancer is glioma.

19. The method of claim 1, wherein the cancer is melanoma.

20. A method of treating an EGFR overexpressing cancer in a mammal, comprising: administering to said mammal therapeutically effective amounts of a compound of formula (I″)

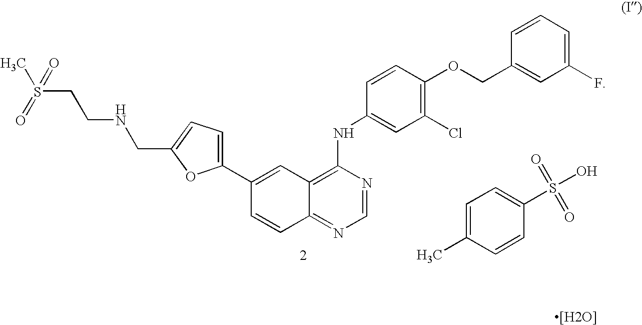

21. A method of treating an erbB2 overexpressing cancer in a mammal, comprising: administering to said mammal therapeutically effective amounts of a compound of formula (I″)

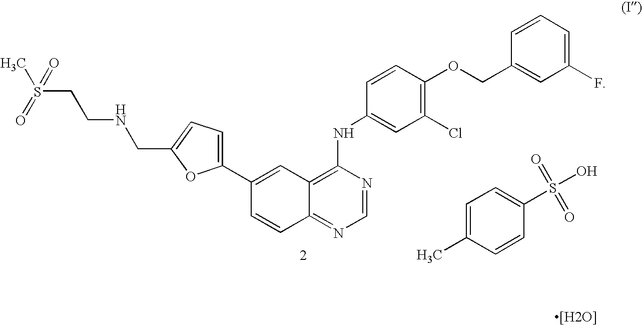

22. A method of treating an EGFR and erbB2 overexpressing cancer in a mammal, comprising: administering to said mammal therapeutically effective amounts of a compound of formula (I″)

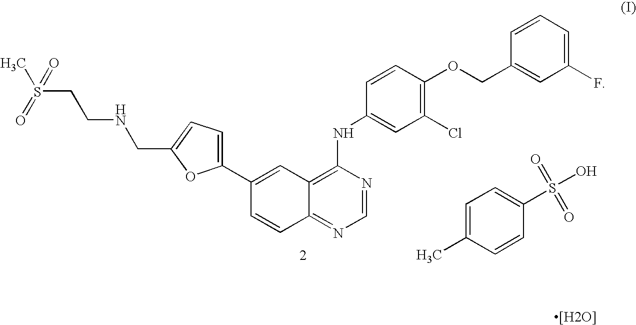

23. A method of treating renal cancer in a mammal, comprising: administering to said mammal therapeutically effective amounts of a compound of formula (I) 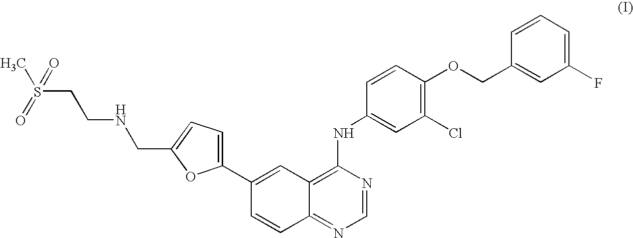
or salts or solvates thereof.

24. The method of claim 23, wherein the compound of formula (I) is a compound of formula (I′) 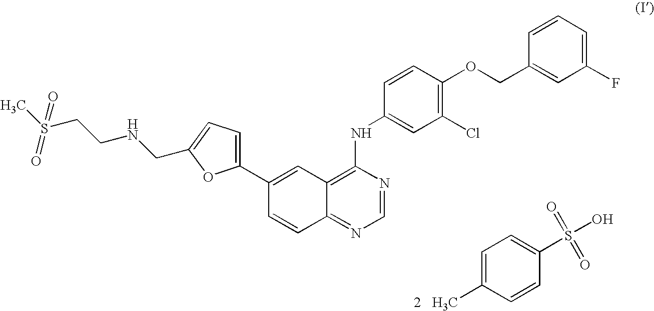
or anhydrous or hydrated forms thereof.

25. The method of claim 23, wherein the compound of formula (I) is a compound of formula (I″)

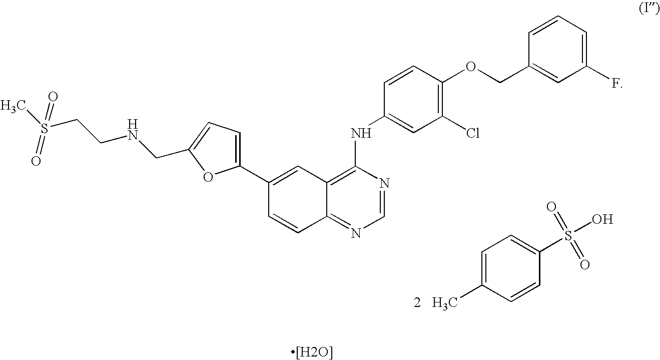

26. The method of claim 23, wherein the renal cancer is renal cell carcinoma.

27. A method of treating urothelial cancer in a mammal, comprising: administering to said mammal therapeutically effective amounts of a compound of formula (I) 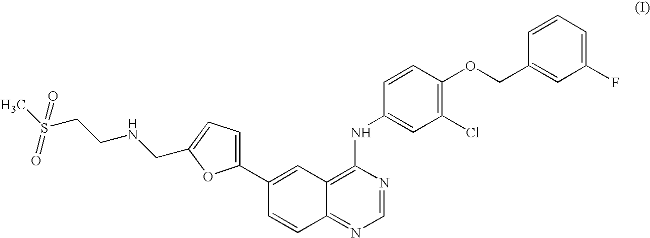
or salts or solvates thereof.

28. The method of claim 27, wherein the compound of formula (I) is a compound of formula (I′) 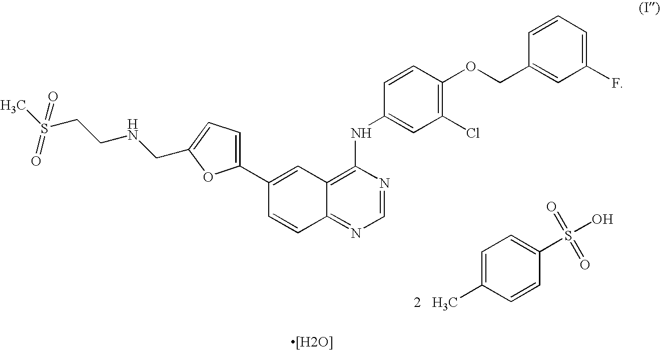
or anhydrous or hydrated forms thereof.

29. The method of claim 27, wherein the compound of formula (I) is a compound of formula (I″)

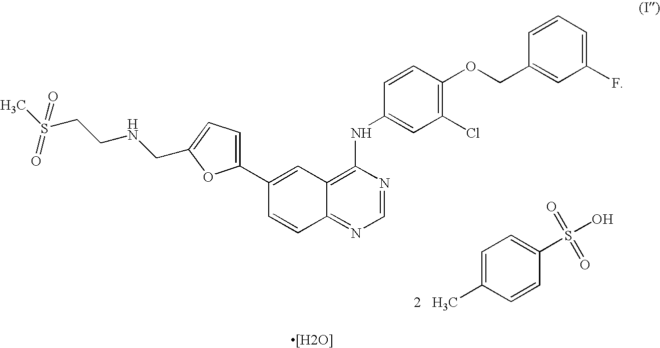

30. The method of claim 27, wherein the urothelial cancer is bladder cancer.

31. The method of claim 27, wherein the urothelial cancer is advanced or metastatic urothelial cancer.

32. The method of claim 27, wherein the urothelial cancer is a transitional cell carcinoma.
